(19) United States
(12) Patent Application Publication
(10) Pub. No.: US 2004/0171598 A1
(43) Pub. Date: Sep. 2, 2004

(54) Use of steroids to treat persons suffering from ocular disorders

(76) Inventors: David P. Bingaman, Fort Worth, TX (US); Abbot F. Clark, Arlington, TX (US); Rajni Jani, Fort Worth, TX (US); Stella M. Robertson, Fort Worth, TX (US)

Correspondence Address: Teresa J. Schultz, Alcon Research, Ltd., 6201 South Freeway, Q-148, Fort Worth, TX, 76124-2099, US

(21) Appl. No.: 10/771,829

(22) Filed: Feb. 4, 2004

Related U.S. Application Data
(60) Provisional application No. 60/448,943, filed on Feb. 20, 2003.

(51) Int. Cl. A61K 31/573

(52) U.S. Cl. 514/179

(57) ABSTRACT

Methods and compositions for treating retinal edema and NPDR are disclosed.

The present invention is directed to the use of steroid formulations for the treatment of persons suffering from retinal edema and/or nonproliferative diabetic retinopathy (NPDR). The steroid formulations may also include the angiostatic agent, anecortave acetate.

### BACKGROUND OF THE INVENTION

Diabetes mellitus is characterized by persistent hyperglycemia that produces reversible and irreversible pathologic changes within the microvasculature of various organs. Diabetic retinopathy (DR), therefore, is a retinal microvascular disease that is manifested as a cascade of stages with increasing levels of severity and worsening prognoses for vision. Some major risk factors reported for developing diabetic retinopathy include the duration of diabetes mellitus, quality of glycemic control, and presence of systemic hypertension. DR is broadly classified into 2 major clinical stages: nonproliferative diabetic retinopathy (NPDR) and proliferative diabetic retinopathy (PDR), where the term “proliferative” refers to the presence of preretinal neovascularization (NV). NPDR encompasses a range of clinical subcategories which include initial “background” DR, where small multifocal changes are observed within the retina (e.g., microaneurysms, “dot-blot” hemorrhages, and nerve fiber layer infarcts), through preproliferative DR, which immediately precedes the development of preretinal NV. Diabetic macular edema can be seen during either NPDR or PDR, however, it often is observed in the latter stages of NPDR and is a prognostic indicator of progression towards development of the most severe stage, PDR.

Macular edema is the major cause of vision loss in diabetic patients, whereas preretinal neovascularization (PDR) is the major cause of legal blindness. NPDR and subsequent macular edema are associated, in part, with retinal ischemia that results from the retinal microvasculopathy induced by persistent hyperglycemia. Data accumulated from animal models and empirical human studies show that retinal ischemia is often associated with increased local levels of proinflammatory and/or proangiogenic growth factors and cytokines, such as prostaglandin E₂, vascular endothelial growth factor (VEGF), insulin-like growth factor-1 (IGF-1), etc. These molecules can alter the retinal microvasculature and cause pathologic changes such as capillary extracellular matrix remodeling, retinal vascular leakage leading to edema, and angiogenesis.

Today, no pharmacologic therapy is approved for the treatment of DR and/or macular edema. The current standard of care is laser photocoagulation, which is used to stabilize or resolve macular edema and retard the progression toward preretinal NV. Laser photocoagulation may reduce retinal ischemia by destroying healthy tissue and thereby decrease metabolic demand; it also may modulate the expression and production of various cytokines and trophic factors. Unfortunately, laser photocoagulation is a cytodestructive procedure and the visual field of the treated eye is irreversibly compromised. Other than diabetic macular edema, retinal edema can be observed in various other posterior segment diseases, such as posterior uveitis, branch retinal vein occlusion, surgically induced inflammation, endophthalmitis (sterile and non-sterile), scleritis, and episcleritis, etc.

Glucocorticoids have been used by the medical community to treat certain disorders of the back of the eye, in particular: Kenalog (triamcinolone acetonide), Celestone Soluspan (betamethasone sodium phosphate), Depo-Medrol (methylprednisolone acetate), Decadron (dexamethasone sodium phosphate), Decadron L. A. (dexamethasone acetate), and Aristocort (triamcinolone diacetate). These products are commonly administered via a periocular injection for the treatment of inflammatory disorders. Because of the lack of efficacious and safe therapies, there is a growing interest in using glucocorticoids for the treatment of, for example, retinal edema and age-related macular degeneration (AMD). Bausch & Lomb and Control Delivery Systems are evaluating fluocinolone acetonide delivered via an intravitreal implant for the treatment of macular edema. Oculex Pharmaceuticals is studying a dexamethasone implant for persistent macular edema. In addition, ophthalmologists are experimenting with intravitreal injection of Kenalog for the treatment of recalcitrant cystic diabetic macular edema and for exudative AMD.

Although glucocorticoids are very effective in treating many ocular conditions, there are significant side effects associated with the available products. Side effects include: endopthalmitis, cataracts, and elevated intraocular pressure (IOP). Although some side effects are due to the glucocorticoid itself, some may result from, or be exacerbated by, excipients in the formulations.

There is a need for glucocorticoid formulations that are effective in treating retinal edema and NPDR while causing no or lessened adverse reactions. The formulations of this invention meet that need.

### SUMMARY OF THE INVENTION

The present application is directed to the treatment of persons suffering from retinal edema or NPDR with a glucocorticoid alone or in combination with anecortave acetate.

### DETAILED DESCRIPTION OF THE PREFERRED EMBODIMENTS

The present invention provides for improved glucocorticoid formulations for the treatment of persons suffering retinal edema (including macular edema and diabetic macular edema (DME)) and NPDR. The formulations provide for reduced side effects by one or more of the following: the absence of certain excipients in the formulations, the concentration of the glucocorticoid, the choice of glucocorticoid, or the method of delivery of the formulation.

Glucocorticoids which may be employed in the present invention include all acceptable compounds which are effective in the treatment of macular edema and/or NPDR. The preferred glucocorticoids include, dexamethasone, fluoromethalone, medrysone, betamethasone, triamcinolone, triamcinolone acetonide, prednisone, prednisolone, hydrocortisone, rimexolone, and pharmaceutically acceptable salts thereof. Further examples of glucocorticoids include prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone. All of the above-cited glucocorticoids are known compounds. Further information about the compounds may be found for example, in The Merck Index, Eleventh Edition (1989), and the publications cited therein, the entire contents of which are hereby incorporated in the present specification by reference.

The compounds are formulated for delivery to the retina for the treatment of edema and/or NPDR. The formulations are purified, non-preserved glucocorticoid formulations. By eliminating preservatives and using at least one purified steroid, such a formulation will eliminate or greatly reduce the incidence of endopthalmitis.

Preferred steroids for treating chronic retinal edema and/or NPDR are less potent than many of the marketed products. For example, prednisolone, prednisolone acetate, rimexolone, fluoromethalone, and fluoromethalone acetate would be useful in such a scenario, but with reduced incidence of cataracts and/or elevated IOP.

The improved formulations can be delivered by intravitreal, posterior juxtascleral, or subconjunctival injection as well as via an implanted device as further below described. All cited patents are herein incorporated by reference.

Particularly preferred implanted devices include: various solid and semi-solid drug delivery implants, including both non-erodible, non-degradable implants, such as those made using ethylene vinyl acetate, and erodible or biodegradable implants, such as those made using polyanhydrides or polylactides. Drug delivery implants, particularly ophthalmic drug delivery implants are generally characterized by at least one polymeric ingredient. In many instances, drug delivery implants contain more than one polymeric ingredient.

For example, U.S. Pat. No. 5,773,019 discloses implantable controlled release devices for delivering drugs to the eye wherein the implantable device has an inner core containing an effective amount of a low solubility drug covered by a non-bioerodible polymer coating layer that is permeable to the low solubility drug.

U.S. Pat. No. 5,378,475 discloses sustained release drug delivery devices that have an inner core or reservoir comprising a drug, a first coating layer which is essentially impermeable to the passage of the drug, and a second coating layer which is permeable to the drug. The first coating layer covers at least a portion of the inner core but at least a small portion of the inner core is not coated with the first coating layer. The second coating layer essentially completely covers the first coating layer and the uncoated portion of the inner core.

U.S. Pat. No. 4,853,224 discloses biodegradable ocular implants comprising microencapsulated drugs for implantation into the anterior and/or posterior chambers of the eye. The polymeric encapsulating agent or lipid encapsulating agent is the primary element of the capsule.

U.S. Pat. No. 5,164,188 discloses the use of biodegradable implants in the suprachoroid of an eye. The implants are generally encapsulated. The capsule, for the most part, is a polymeric encapsulating agent. Material capable of being placed in a given area of the suprachoroid without migration, “such as oxycel, gelatin, silicone, etc.” can also be used.

U.S. Pat. No. 6,120,789 discloses the use of a non-polymeric composition for in situ formation of a solid matrix in an animal, and use of the composition as a medical device or as a sustained release delivery system for a biologically-active agent, among other uses. The composition is composed of a biocompatible, non-polymeric material and a pharmaceutically acceptable, organic solvent. The non-polymeric composition is biodegradable and/or bioerodible, and substantially insoluble in aqueous or body fluids. The organic solvent solubilizes the non-polymeric material, and has a solubility in water or other aqueous media ranging from miscible to dispersible. When placed into an implant site in an animal, the non-polymeric composition eventually transforms into a solid structure. The resulting implant provides a system for delivering a pharmaceutically effective active agent to the animal. According to the '789 patent, suitable organic solvents are those that are biocompatible, pharmaceutically acceptable, and will at least partially dissolve the non-polymeric material. The organic solvent has a solubility in water ranging from miscible to dispersible. The solvent is capable of diffusing, dispersing, or leaching from the composition in situ into aqueous tissue fluid of the implant site such as blood serum, lymph, cerebral spinal fluid (CSF), saliva, and the like. According to the '789 patent, the solvent preferably has a Hildebrand (HLB) solubility ratio of from about 9-13 (cal/cm3)½ and it is preferred that the degree of polarity of the solvent is effective to provide at least about 5% solubility in water.

Polymeric ingredients in erodible or biodegradable implants must erode or degrade in order to be transported through ocular tissues and eliminated. Low molecular weight molecules, on the order of 4000 or less, can be transported through ocular tissues and eliminated without the need for biodegradation or erosion.

Another implantable device that can be used to deliver formulations of the present invention is the biodegradable implants described in U.S. Pat. No. 5,869,079.

For posterior juxtascleral delivery of a formulation of the present invention, the preferred device is disclosed in commonly owned U.S. Pat. No. 6,413,245 B1 (cannula). Other preferred devices for delivery are disclosed in other commonly owned patents and patent applications: U.S. Pat. Nos. 6,416,777 B1 and 6,413,540 B1 (device for implantation on outer surface of the sclera).

Exemplary glucocorticoid formulations which serve the purpose of the present invention are specifically shown below in Examples 1-7. The suspensions may be delivered as previously described. The formulations of the present invention can include other non-ionic surfactants than tyloxapol, e.g., polysorbates, also known as Tweens, pluronics, and Spans. Ionic surfactants can also be used, e.g., sodium lauryl sulfate or anionic bile salts. Amphoteric surfactants, such as, lecithin and hydrogenated lecithin can be used. The pH can vary from 5.0-8.4, but is preferably about 6.8-7.8. Other appropriate buffer systems, such as, citrate or borate can be employed in the present formulations. Different osmolality adjusting agents can also be used, such as, potassium chloride, calcium chloride, glycerin, dextrose, or mannitol.

### EXAMPLE 1

### Triamcinolone Acetonide Sterile Suspension

| Ingredient | Concentration w/v % |
|---|---|
| Triamcinolone Acetonide | 0.4-2.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

### EXAMPLE 2

### Rimexolone Sterile Suspension

| Ingredient | Concentration w/v % |
|---|---|
| Rimexolone | 0.1-4.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

### EXAMPLE 3

### Prednisolone Sterile Suspension

| Ingredient | Concentration w/v % |
|---|---|
| Prednisolone Acetate | 0.1-2.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

### EXAMPLE 4

### Fluoromethalone Acetate Sterile Suspension

| Ingredient | Concentration w/v % |
|---|---|
| Fluoromethalone Acetate | 0.1-1.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

The present invention also contemplates the use of a glucocorticoid in combination with the angiostatic agent, anecortave acetate. As used herein, anecortave acetate refers to 4,9(11)-pregnadien-17α,21-diol-3,20dione-21-acetate and its corresponding alcohol (4,9(11)-pregnadiene-17α,21-diol-3,20-dione). Presently, anecortave acetate is undergoing clinical trials for its use in persons suffering from subfoveal choroidal neovascularization secondary to AMD. The glucocorticoid alone or in combination with anecortave acetate is useful for treating persons suffering from retinal edema and/or NPDR. In addition to being effective in inhibiting the neovascularization associated with progression to PDR, anecortave acetate is useful in controlling any IOP rise associated with the use of a glucocorticoid. The glucocorticoid and anecortave acetate may be formulated and administered as previously described. Additionally, the glucocorticoid may be dosed as previously described and anecortave acetate may be dosed topically.

Examples of formulations of the above-described combination are shown below:

### EXAMPLE 5

| Ingredient | Concentration w/v % |
|---|---|
| Anecortave Acetate | 3% |
| Triamcinolone Acetonide | 0.5-4.0% |
| Monobasic Sodium Phosphate Diltydrate | 0.051% |
| Dibasic Sodium Phosphate Dodecahydrate | 0.5% |
| Tyloxapol | 0.05-0.4% |
| Sodium Chloride | 0.76% |
| NaOH/HCl | pH adjust to 5.0-8.4 |
| Water for injection | q.s. 100% |

### EXAMPLE 6

A typical example of topical formulation of Anecortave Acetate is as follows:

| Ingredient | Concentration w/v % (Preferred Range) | |
|---|---|---|
| Anecortave Acetate | 0.1-6% | (1-3%) |
| Polyquad | 0.0005-0.01% | (0.001%) |
| HPMC | 0.02-1.0% | (0.5%) |
| Mannitol (b) | 0.0-5.0% | (3.82%) |
| Sodium Chloride (d) | 0.0-0.8% | (0.17%) |
| Disodium Edetate | 0.0-0.2% | (0.01%) |
| Polysorbate-80 (c) | 0.005-0.4% | (0.05%) |
| NaOH and/or HCl | q.s. pH 5.0-8.4 | (6.8-7.8) |

| | |
|---|---|
| Purified Water | q.s. 100% |

(a) other suitable polymers include cellulosic polymers like HPMC, HEC, sodium CMC), polyvinyl alcohol (PVA), Polyvinyl Pyrrolidone (PVP), polyacrylamide, and other water miscible/soluble polymers to impart viscosity to the product and to stabilize suspension.

(b) both ionic as well nonionic agents are used to adjust Osmolality of the product either alone or in combination. This also stabilize the suspension.

(c) other surfactants that can be used are non-ionic (Tyloxapol, Tweens, Spans) anionic (lecithin, hydrogenated lecithins), or anionic (sodium lauryl sulfate, bile salts).

### EXAMPLE 7

### Unit Dose Composition (Preservative Free Product Packaged in Unit Dose)

| Ingredients | Concentration (Preferred Range) | |
|---|---|---|
| Anecortave Acetate | 0.1-6% | (1-3%) |
| Carbomer 974P | 0.02-0.8% | (0.3%) |
| Mannitol | 0.0-5.0% | (3.82%) |
| Sodium Chloride | 0.0-0.8% | (0.17%) |
| Polysorbate-80 | 0.005-0.4% | (0.05%) |
| NaOH/HCl | q.s. pH 4.0-8.0 | (6.8-7.8) |

| | |
|---|---|
| Purified Water | q.s. 100% |

### EXAMPLE 8

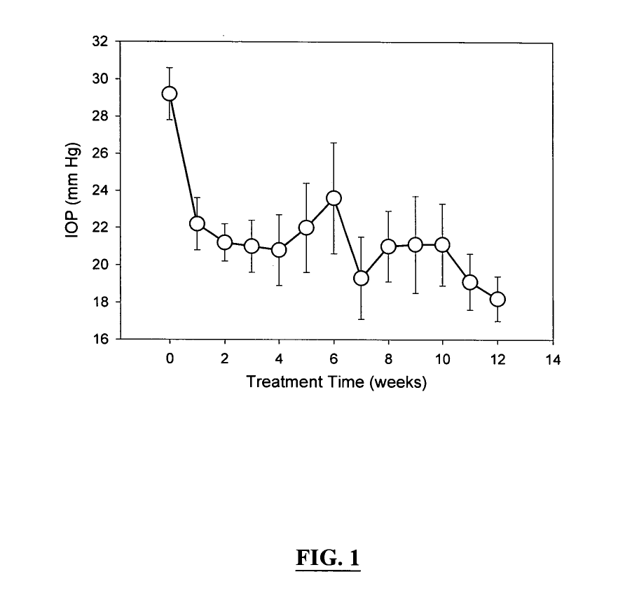

Patients (η=15) with documented glucocorticoid induced ocular hypertension were treated topically with 1% anecortave acetate eye drops three times per day for up to 12 weeks. The patients continued to receive their glucocorticoid medication. IOP was significantly reduced after anecortave acetate treatment (from 29 mm Hg to˜19-22 mm Hg). See FIG. 1.

### EXAMPLE 9

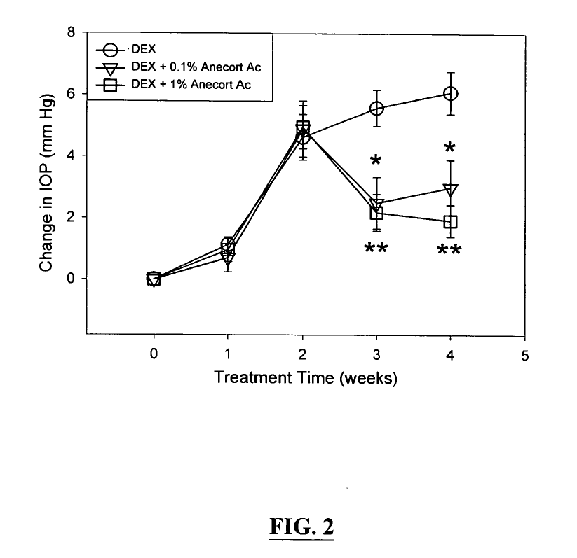

Three groups of rabbits received weekly subTenon's injections of dexamethasone acetate (1 mg/kg) for 4 weeks. After two weeks, the IOPs of all three groups measured approximately 5 mm Hg. The rabbits were then treated with vehicle, 0.1% anecortave acetate, a 1% anecortave acetate by topical ocular dosing three times a day for the remaining 2 weeks. The IOP continued to increase in the vehicle treated group. In contrast, IOP was significantly lowered in both the anecortave acetate treated groups. See FIG. 2.

### EXAMPLE 10

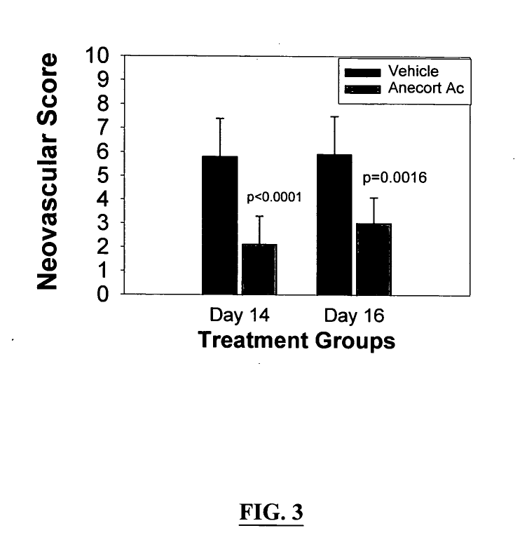

Anecortave acetate was tested for its angiostatic efficacy in a rat pup model of retinopathy of prematurity (Penn, at al., Investigative Ophthalmology & Visual Science, “The Effect of an Angiostatic Steroid on Neovascularization in a Rat Model of Retinopathy of Prematurity,” Vol. 42(1):283-290, January 2001). Newborn rat pups were placed in an atmosphere of varying oxygen content. The rats received a single intravitreal injection of vehicle or anecortave acetate (500 μg) upon return to room air (day 14) or 2 days later (day 16). There was significant retinal neovascularization in the rats that receive vehicle injections. Anecortave acetate significantly inhibited retinal neovascularization by 66% and 50% on days 14 and 16 (respectively). See FIG. 3.

We claim:

1. A method for treating a person suffering from retinal edema or non-proliferative diabetic retinopathy which comprises, administering an effective amount of a formulation free of classical preservatives and comprising a glucocorticoid.

2. The method of claim 1 wherein the formulation further comprises an effective amount of anecortave acetate.
